(19) United States
(12) Patent Application Publication
(10) Pub. No.: US 2023/0080344 A1
(43) Pub. Date: Mar. 16, 2023

(54) SYSTEMS AND DATABASES FOR SOCIAL MEDIA APPLICATIONS

(71) Applicant: APP CO 1, LLC, Beverly Hills, CA (US)

(72) Inventors: Alex Nahai, Los Angeles, CA (US); Andrew Read, London (GB)

(21) Appl. No.: 17/736,050

(22) Filed: May 3, 2022

Related U.S. Application Data
(60) Provisional application No. 63/243,365, filed on Sep. 13, 2021.

(51) Int. Cl. G06Q 50/00; G06F 16/735

(52) U.S. Cl. CPC G06Q 50/01; G06F 16/735

(57) ABSTRACT

A system for displaying user generated content files to a group of users is provided. The system divides the users into groups based on mental health conditions and then selectively shows the user generated content to the groups based on matching tags on the user generated content. A comorbidity cross-reference is used to cross-reference tangentially related content to users not directly interested.

### CROSS-REFERENCE TO RELATED APPLICATIONS

This application claims the benefit of U.S. Provisional Application No. 63/243,365 filed Sep. 13, 2021, which is incorporated herein in its entirety.

### FIELD

This patent document relates to social media applications and in particular improved systems and databases for social media applications. In preferred embodiments, the systems and databases are used in connection with social media applications for the benefit of mental health. In other aspects, this patent document relates to new methods and systems for engaging users on a social media application and in particular, with regards to mental health issues.

### BACKGROUND

Social Media applications are known in the art. In particular, Instagram®, Facebook® and TikTok® are all social media applications that have a large pool of users and user content and have to decide what content is pertinent to show what users.

The ability to show users content that those users find interesting is likely the number one distinguishing factor for social media application success. If users are shown content they are not interested in, the users will quickly become disinterested and engagement of the social media application will suffer. In contrast, if users are shown new content they are interested in, the users will continue to engage with the social media application and the social media application will flourish.

In most social media applications, users create content and upload it to servers owned by the social media company. The social media companies must store and organize all this uploaded content so that it is easily searchable and accessible to social media applications. If the search takes too long, the user experience will suffer. Accordingly, it is very important that the systems and databases used to store content created by users can be easily searchable and related content is quickly findable. Moreover, it is important that the social media application can determine which content each individual user may find interesting and which content will not be of interest to a particular user. Without this ability, the entire social media application will fail.

There are very few websites, systems and methods available for addressing mental health issues online. To this end, a social media application particularly suited for addressing mental health issues would be beneficial. In order to allow such a social media application, new and advanced systems and databases are needed that allow content related to mental health to be quickly searchable and selected such that pertinent content can be provided to users on a timely basis. To this end, it would be beneficial to have databases that associate content, especially mental health content, in new ways.

Moreover, there is a need for new and improved methods and systems for determining whether to distribute content to a particular user from a database of user content. While the systems and methods discussed herein are not to be limited to the areas of mental health, they are particularly adapted for use with a mental health social media application.

### SUMMARY OF THE EMBODIMENTS

Objects of the present patent document are to provide new and improved systems and methods for engaging users on a social media application. To this end, a system for displaying user generated content files, such as video files or short form video files, is provided. In preferred embodiments, the system for displaying user generated content comprise a plurality of user profiles stored in a user database wherein each user profile in the plurality of user profiles is associated with one or more user mental health conditions.

The system includes a first server that for each user mental health condition creates at least a first group of user profiles that are user profiles associated with the user mental health condition and a second group of user profiles that are user profiles that are not associated with the mental health condition.

The system further comprises a network of servers with a plurality of user generated content files stored thereon, wherein each user generated content file in the plurality of user generated content files is associated with one or more tags that classifies each user generated content file with the one or more mental health conditions.

The system may comprise a second server that for each tag in the one or more tags classifies each user generated content file in the plurality of user generated content files into the first group of user profiles or the second group of user profiles based on whether a tag in the one or more tags matches the user mental health condition.

The system has a comorbidity database with a plurality of comorbidity database entries wherein each comorbidity database entry in the plurality of comorbidity database entries includes a comorbidity cross-reference that cross references the one or more tags of a first subset of user generated content files associated with the first group of user profiles with a subset of user profiles from the second group of user profiles that have tangentially related mental health conditions to the one or more tags of the first subset of user generated content files.

A third server may be configured to publish the first subset of user generated content files to the subset of user profiles. As may be appreciated, more or fewer servers may be used in various different embodiments.

In preferred embodiments, the plurality of user generated content files are video files. Even more preferably, the video files are short form video files between five seconds and ten minutes in length. In even more preferred embodiments, the video files are short form video files between five seconds and two minutes in length.

In some embodiments, engagement of the first subset of user generated content files with the subset of user profiles is a factor of the comorbidity cross-reference. In some embodiments, a frequency that a first tag of a first mental health condition is used in a single user generated content file with a second tag of a second mental health condition is a factor of the comorbidity cross-reference.

Prioritizing user generated content is also important. In some embodiments, a level of engagement of the first subset of user generated content files within the first group of user profiles is used to prioritize the first subset of user generated content files to the subset of user profiles. In some embodiments, demographics are used to prioritize the first subset of user generated content files to the subset of user profiles. In still yet other embodiments, recency is used to prioritize the first subset of user generated content files to the subset of user profiles.

### BRIEF DESCRIPTION OF THE DRAWINGS

FIG. 1 illustrates a system for selectively showing a plurality of user generated content files;

FIG. 2 illustrates a mobile device that includes one embodiment of the client-side application;

FIG. 3 illustrates one embodiment of a home screen for a user of the social media application running on a mobile device;

FIG. 4 illustrates one embodiment of a screen to allow users to select topics of interest to associate their profile with;

FIG. 5 illustrates one embodiment of a screen that allows a user to select from a plurality of mental health condition tags;

FIG. 6 illustrates one example of the complicated data structures that are created by the application for all the users and the various groups.

### DETAILED DESCRIPTION OF THE DRAWINGS

The following detailed description includes representative examples utilizing numerous features and teachings, both separately and in combination, and describes numerous embodiments that relate to systems, databases, methods, and machine-readable mediums for use with social media applications. This detailed description is merely intended to teach a person of skill in the art further details for practicing one or more embodiments of the present disclosure and is not intended to limit the scope of the claims. Therefore, combinations of features disclosed in the following detailed description and incorporated documents may not be necessary to practice the teachings in the broadest sense, and are instead taught merely to describe particularly representative examples of the present teachings.

Some portions of the detailed description that follows are presented in terms of algorithms and sequences of operations, which are performed within a computer memory. These algorithmic descriptions and representations are the means used by those skilled in the data processing arts to most effectively convey the substance of their work to others skilled in the art. An algorithm or sequence of operations is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated.

It should be borne in mind, however, that the algorithms and/or sequence of operations are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic device, that manipulates and transforms data represented as physical (electronic) quantities within the computer system's registers and memories into other data similarly represented as physical quantities within the electronic device's memory or registers or other such information storage, transmission or display devices.

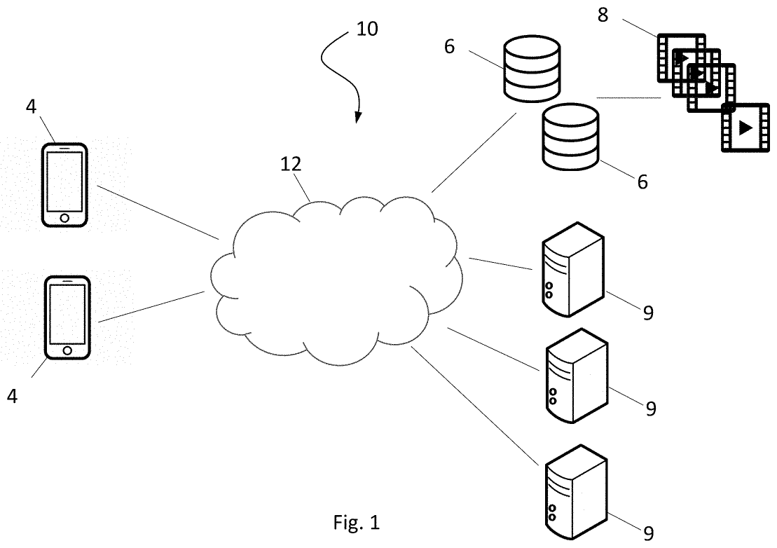

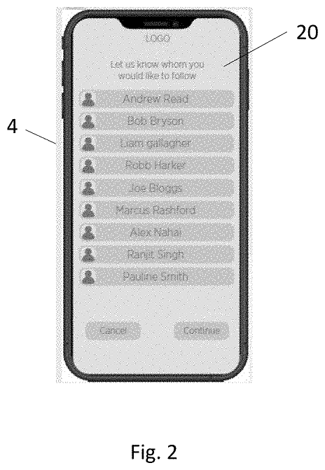

FIG. 1 illustrates a system 10 for selectively showing a plurality of user generated content files 8. Systems 10, databases 6 and methods geared toward self-improvement and personal growth in connection with mental health are provided herein. In preferred embodiments, the client application is embodied in a mobile application for use with a mobile device 4 such as a cellular phone or tablet. FIG. 2 illustrates a mobile device 4 that includes one embodiment of the client-side application 20.

Returning to FIG. 1, the server-side application(s) are embodied in one or more applications and databases 6 or programmable databases 6 running on servers 9. However, in other instances, the applications may be viewed through a website over the internet 12 used on a personal computer or other device. In still yet other embodiments, other types of applications or devices may be used.

In preferred embodiments, the server side and client-side application(s) work together to create a social media-type application based on a plurality of user generated content files 8. The user generated content files 8 are, as the name implies, content created by the users of the social media application and uploaded to the social media platform. In preferred embodiments, the user generated content files 8 are media files such as video, photographs, music, artwork or a combination of any of these. In even more preferred embodiments, the user generated content files 8 are short video content generated by users.

As used herein, the phrase “short video content” means videos between five seconds and ten minutes. In more preferred embodiments, videos between five seconds and five minutes may be used. In more preferred embodiments, videos between five seconds and two minutes may be used. In more preferred embodiments, videos between five seconds and one minute may be used.

In preferred embodiments, the social media application would manage millions or more user generated content files 8. Accordingly, a network of servers 9 is required to store all the user generated content files 8. The servers are all linked via a network 12, which may be the internet or a local network. In preferred embodiments, the servers are using databases 6 to keep track of the user generated content files 8 and other data important to the social media application.

As may be appreciated, when a user joins the social media application, a user profile is created and stored by the server 9. As is known, user profiles may be linked to other user profiles within the application. This is sometimes known as friending, following, connecting or supporting. As may be seen in FIG. 2, when a user creates a user profile, they may be asked to follow other user profiles they may know. As may be appreciated, content posted by a user that is being followed may appear in the following user's content feed. FIG. 2 illustrates one embodiment of a client application that shows a screen asking the user to select other users he/she may know to follow.

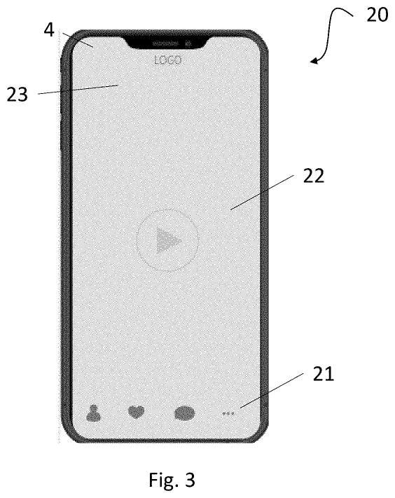

FIG. 3 illustrates one embodiment of a home screen for a user of the social media application running on a mobile device 4. The home screen has a content viewing area 22 in which the user generated content files 8 may be displayed. In preferred embodiments, the user generated content files 8 may be shared by users, liked by users and commented on by users. To this end, the embodiment 20 shown in FIG. 3 has a button with three dots 21 that when selected, pop out in order to allow the user to perform a number of interactions including without limitation, Appreciating, Commenting, Sharing or Reporting another user's content. As one skilled in the art may appreciate, any number of user interactions and ways to interact may be performed by on or with the content files by the users of the social media application.

In preferred embodiments, the areas of focus of the application are to be issues that are most commonly seen in either clinical therapy practice or in counseling/life coaching practice. However, the teachings herein can be used with any topic. Because many issues in life at some point may involve an impact on one's emotional/mental state, the following are just some examples of topics that users may generate user generated content for: 1) Strictly Mental health-based content; 2) Physical health-based content; 3) Relationship-based content; 4) Profession/career/personal financial-based content; 5) Addiction-based content; and 6) Identity-based content.

In preferred embodiments, the user experience on the social media application will not have a clinical feel. One objective of the application is to de-stigmatize and normalize sharing experiences pertaining to mental health.

Some goals of the embodiments herein are to create an environment for people to feel comfortable sharing content and supporting their peers with respect to the aforementioned topics, to create meaningful friendships and relationships, and to give users better insights into the lives of their peers and loved ones.

Other goals include to change the existing social media paradigm, and become the designated space for people to build connections based on real life experiences, events, and conversations.

The applications discussed herein are the first designated social media platforms for people desirous of self-improvement in the mental health arena to be able to share their experiences and develop tools for achieving this growth. The applications discussed herein provide a place where people are not encouraged to manufacture an image of perfection, but to open up about their personal issues (past or present) and aspirations (whether specific or general). Another goal of the applications and methods discussed herein is to catalyze the deterioration of any stigma associated with opening up about difficult life issues.

The purpose of the systems the application uses is to deliver bespoke user generated content based on an individual's indicated interests with respect to mental health. Another objective of the algorithms is to deliver content that is tangentially related to the interests of the user by linking content through a comorbidity connection.

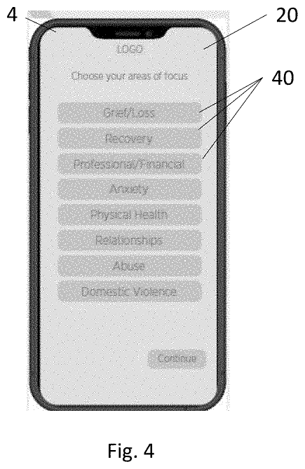

In preferred embodiments, users may provide information when they create their user profile, or at any time thereafter, about the topics, issues, or mental health condition they have an interest in. FIG. 4 illustrates one embodiment of a screen to allow users to select topics of interest to associate their profile with such as Mental Health, Grief/Loss, Recovery, Addiction, Professional/Financial, Physical Health, Relationships, Abuse and Domestic Violence to name just a few.

In preferred embodiments, users are categorized into groups with respect to a plurality of mental health conditions. The users may be categorized by a server 9 or more specifically by an application running on a server 9. As used herein, the term “mental health conditions” includes not only actual diagnosis like bipolar disorder, attention deficit disorder or schizophrenia but also includes symptoms like anxiety or lack of energy. As may be appreciated, there may be some crossover with these tags. For example, anxiety may be a diagnosis in some instances but a symptom in others.

Users may be categorized into more than one group for each mental health condition and may be categorized into groups associated with more than one mental health condition. In preferred embodiments, users are categorized into at least being in or out of the following groups with respect to each mental health condition: Sampling Group, Interested Group and Interim Group. In preferred embodiments, in addition to being categorized into at least being in or out of the aforementioned groups, users are all considered to be in the General Population of the application.

In the simplest embodiment, user profiles are categorized into at least two groups. The first group is comprised of those users interested in a mental health condition and the second group is comprised of all the user profiles not interested in a particular mental health condition.

In order to determine whether a user is going to be a member of any of the groups associated with a particular mental health condition, a number of factors may be used. As non-limiting examples, any of the following can be used:
Data: actual, concrete data points that users have entered into the application at any time while using the application, including, without limitation, during the process of joining application, when posting content or when interacting with other users such as selecting any of the topics shown on the screen in FIG. 4.
Behavior: users' behaviors as remembered by the application in connection with the way that users interact with content they prefer or with individuals that they have built connections with on the application.
Demographics, which can help inform priority of content once a user has been identified as an interested user.

To this end, the system includes a plurality of user profiles. The user profiles are preferably stored in a user database 6 wherein each user profile in the plurality of user profiles is associated with one or more user mental health conditions. This may be done by using tags, columns in the database, or a data structure that contains the mental health conditions and associates them with the user profile.

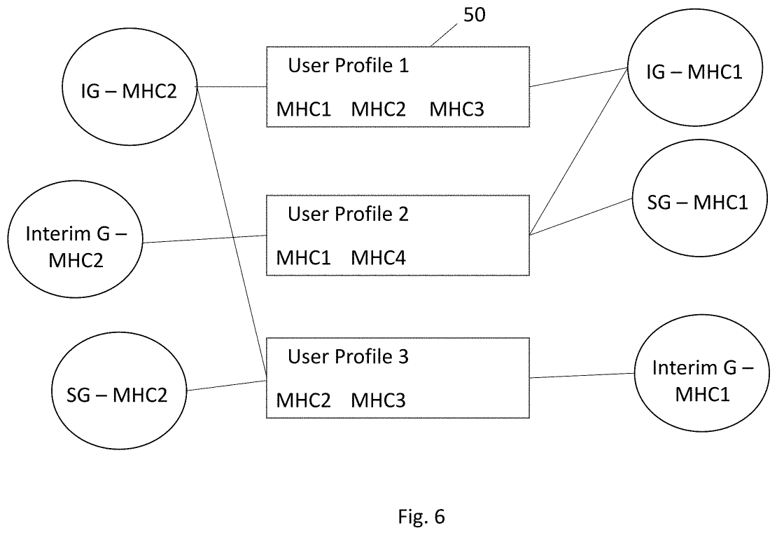

FIG. 6 illustrates one example of the complicated data structures that are created by the application for each piece of user generated content on the platform.

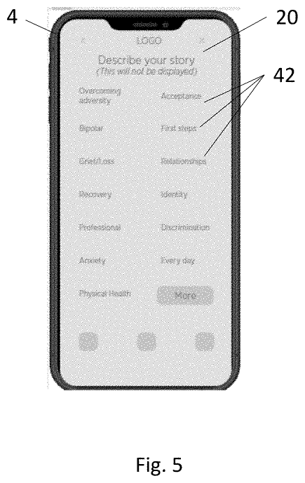

In order to ensure that each piece of content that circulates amongst the main feeds of the users is interesting and captivating, users will be asked to tag each piece of user generated content they post according to one or more areas of interest within the mental health space. FIG. 5 illustrates one embodiment of a screen that allows a user to select from a plurality of tags 42. The one or more tags 42 will then be associated with the posted user generated content 8. Tags 42 can be saved as meta data with the user generated content and/or can be saved in a database that associates the user generated content with the tag, for example, as a record in a table. In yet other embodiments, a data structure may be created that includes the user generated content 8 or a pointer to the user generated content and associates the user generated content 8 with the one or more tags 42.

Tags 42 may be in a number of categories to help categorize user content. For embodiments dealing with mental health, one group of tags may be all related to actual mental health conditions, for example, bipolar disorder, schizophrenia or depression. Another category for tags might be symptoms such as anxiety or fatigue. More than one tag and more than one tag in a particular category can be associated with any user generated content file by the user. As may be appreciated in FIG. 6, tags 42 may include, but are not limited to: overcoming anxiety, lessons learned, grief/loss, professional, mental health, physical health, acceptance, first steps, relationships, identity, discrimination, every day and more.

The social media application then performs a comparison of the one or more tags associated with the user generated content 9 and compares those tags with interests associated with each user's profile. To this end, the application can classify the users on the application into the Sampling Group, Interested Group, Interim Group for each tag/mental health condition.

FIG. 6 illustrates one example of the complicated data structures that are created by the application for all the users and the various groups. In the illustration in FIG. 6, there are three user profiles 50 that have each been associated with various mental health conditions shown as MHC1, MHC2 . . . MHC4. As seen in FIG. 6, user profiles 1 and 2 are both in the Interested Group for MHC1 because they both include that tag MHC1. User profile 2 is in the Sampling Group. Finally, only user profiles with established tangentially related interests are in the Interim Group. A similar data structure is created for user profile 2 and 3. To this end, each mental health condition, MHC1-MHC4 has three groups of user profiles associated with it.

When a piece of user generated content is added, it is tagged by the user and thus, the server, or an application running on a server, knows which user profiles to begin showing the user generated content to. Accordingly, user generated content files are shown to various groups (i.e. Sampling Group, Interested Group, Interim Groups) for each mental health condition. The application maps each user generated content file 9 into three user groups for each condition.

The Sampling Group is the smallest group and the first group to get to validate the quality of the user generated content. The Sampling Group is comprised of users whose user profile interests match with at least one tag in the user generated content but is as the name suggests, just a sampling of those users.

The Sampling Group may be thought of as a type of content validation process. One purpose of the Sampling Group is to be a gatekeeper to filter out undesirable, poorly engaged, or potentially harmful/triggering, content. The Sampling Group can serve as a filtration process for the Content Based Channel. The Content Based Channel refers to content that will appear in a user's feed that is not posted by someone the user follows. Users who are shown content as part of the Sampling Group will also be given the opportunity to report harmful or triggering content, or content that is violative of the application's community standards, and therefore, the Sampling Group will also serve as a process to reinforce a safe environment on the application. Content on the application that is initially displayed to the Sampling Group will also be analyzed by the application's Artificial Intelligence and/or Machine Learning systems and removed if it is deemed harmful, triggering, or violative of the application's community standards. Each piece of user generated content that is posted on the application goes through a filtration process that involves the Sampling Group. In preferred embodiments, content posted by an account followed by a user is shown to that user and is not subject to any filtration process.

In preferred embodiments, the users that are part of the Sampling Group for any given piece of user generated content are chosen at random from users whose profiles have known interests in mental health related conditions or topics that match at least one tag on the user generated content. As will be understood for the explanation of the Interested Group that follows, the Sampling Group is a small subset of the users in the Interested Group. The Sampling Group is a percentage of arbitrary users with matching interests to those tagged in the content/video by the user that generated it.

In preferred embodiments, the Sampling Group may be 10% of the Interested Group but should not be less than ten people. In other embodiments, the Sampling Group may be a lower percentage of the Interested Group, especially as the total number of users increases. In some embodiments, the size of the Sampling Group may be capped at a number such as five thousand users in the same way the minimum may be ten. As one skilled the art will recognize, the size of the cap and limit may be adjusted as necessary and also depending on the size of the total population of the application.

If engagement in the Sampling Group is above a threshold, then the user generated content is shown to the larger Interested Group. The Interested Group is every user whose interests match with at least one tag of the user generated content. If engagement in the Interested Group is above a threshold, then the user generated content may be shown to an Interim Group. The Interim Group is comprised of users whose interests do not have an exact match with any of the tags of the user generated content but through correlation process explained in more detail below, are selected to see the user generated content.

A single piece of content may be tagged with more than one mental health disorder or symptom and thus, there may be a plurality of Sampling Groups, Interested Groups and Interim Groups for any individual piece of content. For example, if a video was tagged with anxiety and bipolar disorder, the social media application would match the video with the Sampling Groups, Interested Groups and Interim Groups of both anxiety and bipolar disorder. That user generated content will then be shown to a small subset of those users who have indicated they are interested in content similar to the subject of the video (“Sampling Group”) based on the tags.

Amongst the Sampling Group, if less than a certain percentage of users meaningfully engage with the video, the video is not shown on anyone else's main content feed, and is limited to the poster's profile page.

Between 5% and 15% of the Sampling Group, and preferably ten percent (10%) of people, are needed to engage with the video in some fashion in order for the video to be shown to the larger interested group of that category, for example, mental health disorder or symptom.

Without limitation, the following are some examples of ways a user may interact with the user generated content in order to demonstrate said engagement: 1) For video, watching past the video preview; 2) For video, watching until the end of the video; 3) Tapping the “Appreciate”, “Like” or other button indicating acceptance linked to the user generated content; 4) Commenting on the user generated content; 5) Adding the user generated content to a user's section of saved content in the application; 6)) Sharing the user generated content within the application with other users; 7) Sharing the user generated content outside the application with other individuals.

If more than the upper threshold percentage (i.e., between 5% and 15% and preferably 10%) of users in the Sampling Group engage with the user generated content, then the user generated content is eligible to be shown to all people with the same interests in their main feeds (the “Interested Group”).

In the event that the upper threshold percentage is met, the user generated content will be available to be shown to all interested users, but the priority with which an interested user will be shown the user generated content will be determined by a weighting system, relative to other content available to be shown to the same user. Accordingly, every piece of content that makes it beyond the content validation system (i.e. sampling group) is eligible to be shown to a user as “interested group content” but whether it is actually shown depends on the other content that is in the user's queue of main feed content, and how it ranks up against those other videos along with how much content the particular user consumes. The more content a user consumes, the farther down in the ranking of content a user will get.

Amongst the Interested Group, if less than a certain percentage of users engage with the video, then the video is only shown to people in the Interested Group. The bottom percentage, between 75% and 85% (and preferably 80% in terms of engagement) of content that is shown to the Interested Group and meaningfully engaged with, will remain only available to be shown to the Interested Group. If more than a certain percentage of users engage with the content (between 15% and 25% and preferably 20% in terms of engagement), then the content is available to be shown to people who have interests that are tangentially related to the interests the content has been tagged with (the “Interim Group”).

The system includes a server running a database that tracks relationships between different mental health conditions and/or different user generated content files. The database may be referred to as a comorbidity database. The comorbidity database includes a plurality of entries wherein each comorbidity database entry in the plurality of comorbidity database entries includes a comorbidity cross-reference. In preferred embodiments, the comorbidity cross-reference cross references user generated content files with user groups for a particular mental health condition that are not the same as the mental health condition of the particular tag being cross referenced. To this end, the comorbidity database can create relationships between user generated content and user groups that are only tangentially related based on their mental health condition and/or tags.

In preferred embodiments, a tag of a particular mental health condition from a piece of user generated content may be an input to a function call to the comorbidity database and the database would return a reference to one or more groups of users associated with a mental health condition that is not the same as the particular mental health condition referenced by the tag. In preferred embodiments, the reference may be in the form of one or more pointers.

The comorbidity database cross-references only tags or mental health conditions that are not the same. In some embodiments, the tag or mental health condition may be an input to a function call to the comorbidity database and the comorbidity database would return a list of other tags or mental health conditions not the same as the input but that the database determines are somehow related. These output tags may then be used to determine the appropriate tangentially related user groups to show the user generated content to.

As may be appreciated, the comorbidity database is used to determine whether a particular piece of user generated content is tangentially related to a group of users without a direct interest in one of the mental health conditions tagged in that piece of content. The group of users created by the comorbidity database is generally called the Interim Group. The comorbidity database may use any number of factors to determine tangentially related users to place in the Interim Group. In preferred embodiments, the following five factors may be used: 1.) Interest and Strength of Connection to co-morbidity; 2.) Frequency; 3.) Engagement; 4.) Demographics; 5.) Recency. In preferred embodiments, the first factor is used to determine tangentially related users to place in the interim group and factors 2-5 are used to help determine the priority of Interim Group content once a piece of content makes it into the Interim Group. The Database informs the strength of connection of one condition to a number of co-morbidities.

In preferred embodiments, the factors may be used in weighted systems such that each factor used adds or subtracts from a weighted value that determines the cross-reference strength of a piece of user generated content to a user profile. Each piece of user generated content can be provided a weighted value based on its various factors to allow the relative strength of correlation of two different pieces of user generated content to a particular user profile.

In some embodiments, the application may use the popularity or success of tangentially related mental health condition within a user group as a factor in determining whether a piece of user generated content is tangentially related to another user group. This is a type of feedback loop such that the application continually refines what content should be shown.

The interest may be determined through a survey of what types of content are commonly chosen together by people on the application (i.e. anxiety and depression). This may be done by analyzing data from use of the application over time to build general correlations and even correlations specific to a user. As the application becomes more populated, data on the way co-morbidities are expressed on the application can be used, further refining the application's understanding of connections between various conditions. In some embodiments, a combination of both may be used such that content is first correlated based on application-wide trends and then customized based on individual user trends.

In addition to interest, strength of connection to co-morbidity may be used. The strength of the co-morbidity connection may be calculated by gleaning possible correlations from data within the application. For example, research shows that “about 78% of adults with Borderline Personality Disorder also develop a substance-related disorder or addiction at some time in their lives” (https://www.ncbi.nlm.nih.gov/pmc/articles/PMC4010862/). Therefore, it is likely that a large percentage of users seeking videos about Borderline Personality Disorder will also seek videos about substance abuse and addiction, thereby indicating a strong correlation and comorbidity between the two disorders. As such, the strength of connections gleaned from data within the application will be representative of the data in the body of research from the medical and psychological communities. Another similar example may be found in the correlation between Body Dysmorphic Disorder and its multiple comorbidities (major depressive disorder, social anxiety disorder, obsessive-compulsive disorder, and substance use disorders)(https://oxfordmedicine.com/view/10.1093/med/9780190254131.001.0001/med-9780190754131-chapter-11). While it is known that these are common comorbidities with Body Dysmorphic Disorder, tracking the number of users with BDD who are seeking and watching videos related to the comorbid disorders mentioned above will provide us with anecdotal data on the strength of the connections for the application's user population.

In addition to data from within the application, other factors that may be considered in determining the strength of comorbidity include, without limitation, clinical research of these conditions, clinical experience of a clinical psychologist to identify connections between mental health conditions, the Diagnostic Statistical Manual (“DSM”), clinical studies, practical application in clinical practice, and other real-world factors and variables that may also be used to understand correlations. Tables may be created that correlate mental health conditions based on clinical understanding and these tables

Through talking with doctors and accessing available clinical research, it is possible to match many mental health disorders and conditions normally present with certain co-morbidities. Through understanding and drawing these correlations between different mental health conditions, content may be presented to individuals that is tangentially related to the interests they have indicated on the application.

Another factor that may be used to determine what user profiles are tangentially related to a user generated piece of content include user behavior and data entry on the application (e.g. how often does one user who has demonstrated an interest in anxiety engage with depression related content once presented with that content? Or how often do user's tag videos with two conditions that are different but possibly related as co-morbidities?).

Another factor that may be used to determine what user profiles are tangentially related to a user generated piece of content include the frequency with which an individual user tags the two related conditions simultaneously when preparing to post a video, or the frequency with which the users on the application demonstrate that they possess certain co-morbidities by posting content about two related conditions.

Engagement may be another factor that determines whether a piece of user content should be tangentially related to a group of users. In certain situations, a particular piece of content may not be correctly tagged by a user or may include content the user is unaware matches another type of mental health condition. To this end, the level of engagement by a particular user group may identify a link in comorbidity. For example, if a video gets a lot of engagement by users that consistently associated with another mental health condition, that may indicate that the particular video is tangentially related to that user group. This may be tracked by the application.

Demographics are another factor that may be considered in determining whether a piece of user generated content is tangentially related to a user group. However, in preferred embodiments, demographics is not used to determine whether a piece of user generated content is tangentially related to a user group but instead is used as a factor in considering how to prioritize content that has already been determined to be tangentially related.

Recency may be an additional factor in whether to link a particular piece of user generated content with a user group. However, in preferred embodiments, recency is not used to determine whether a piece of user generated content is tangentially related to a user group but instead is used as a factor in considering how to prioritize content that has already been determined to be tangentially related. Because the application needs to continue to show relevant content to keep user engagement up, the recency of a video submission may be a factor in determining whether to show a video to tangentially related users or the priority in which to show a video. If a video is too old, it may not be considered tangentially related even if it scores well in other categories or it may be prioritized lower on a user's feed. In preferred embodiments, the strength of the recency factor may fall off exponentially with age.

The top percentage of content (between 15% and 25% and preferably 20%) that is shown to the Interested Group and meaningfully engaged with will be shown to the Interim Group. Content in the Interim Group may also be throttled for each medical condition. For example, tangentially related content shown in the Interim Group may be tracked to each user and indicators kept. The Interim Group system would not show a user a video on the same condition more than once until at least one of each strongly correlated comorbid conditions have been displayed. If two videos are similarly engaged with, then demographic similarities are applied as the next tie breaker. If both engagement and demographics are equal between two videos then priority is determined based on recency with which content is posted.

All the content available to be shown to an Interim Group may be prioritized. In preferred embodiments, engagement is the main factor to prioritize content to the Interim Group. User generated content that is meaningfully engaged within the Interested Group may be prioritized within the Interim Group.

Amongst the Interim Group, if less than a certain percentage of users engage with the video, then the video is only shown to people in the Interim Group.

The bottom percentage of content, between 75% and 85% and preferably 80%, that is shown to the Interim Group and meaningfully engaged with will remain only shown to the Interim Group. If more than a certain percentage (15% and 25% and preferably 20%) of users from the Interim Group engage with a video that graduated from the Interested Group to the Interim Group, then the video is shown to people generally on the app, whether or not they have delineated that they are interested in content that corresponds to what category(ies) the poster tagged the video with (the “General Population”).

The top percentage of content, between 15% and 25% and preferably 20%, that is shown to the Interim Group that graduated from the Interested Group and is meaningfully engaged with will be shown to the General Population. One type of content that is shown to the General Population will be “popular” content that has highest amongst the highest levels of engagement of any videos posted to the application within a six-month retroactive time period, and another type of content that is shown to the General Population will be “trending” content that has amongst the highest engagement of any videos posted to the application within a one-week retroactive time period.

Within all of the aforementioned groups of users, the order of priority with which each user is displayed content will depend on a complex weighting system. The weighting system examines all of the different types of engagement on the application and ranks them according to strength of engagement. Based on a combination of hundreds of variables, content recommendations are ranked. Through this weighting system, a unique order of priority for content displayed to each user is determined.

It should also be understood that a variety of changes may be made without departing from the essence of the invention. Such changes are also implicitly included in the description. They still fall within the scope of the invention. It should be understood that this disclosure is intended to yield a patent or patents covering numerous aspects of the invention both independently and as an overall system and in both method and apparatus modes.

Further, each of the various elements of the invention and claims may also be achieved in a variety of manners. This disclosure should be understood to encompass each such variation of an embodiment of any apparatus embodiment, systems, databases, methods, machine-readable medium or process embodiment, or even merely a variation of any element of these.

Particularly, it should be understood that as the disclosure relates to elements of the invention, the words for each element may be expressed by equivalent apparatus terms of method terms—even if only the function or result is the same. Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action. Similarly, each physical element disclosed should be understood to encompass a disclosure of the action which that physical element facilitates.

Any patents, publications, or other references mentioned in this application for patent are hereby incorporated by reference. In addition, as to each term used, it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans, incorporated herein by reference.

Further, all claim terms should be interpreted in their most expansive forms so as to afford the applicant the broadest coverage legally permissible. Although the embodiments have been described with reference to the drawings and specific examples, it will readily be appreciated by those skilled in the art that many modifications and adaptations of the processes, systems and databases described herein are possible without departure from the spirit and scope of the embodiments as claimed herein. Thus, it is to be clearly understood that this description is made only by way of example and not as a limitation on the scope of the embodiments as claimed below.

1. A system for displaying user generated content files comprising:
a plurality of user profiles stored in a user database wherein each user profile in the plurality of user profiles is associated with one or more user mental health conditions;
a first server that for each user mental health condition creates at least a first group of user profiles that are user profiles associated with the user mental health condition and a second group of user profiles that are user profiles that are not associated with the mental health condition;
a network of servers with a plurality of user generated content files stored thereon, wherein each user generated content file in the plurality of user generated content files is associated with one or more tags that classifies each user generated content file with the one or more mental health conditions;
a second server that for each tag in the one or more tags classifies each user generated content file in the plurality of user generated content files into the first group of user profiles or the second group of user profiles based on whether a tag in the one or more tags matches the user mental health condition;
a plurality of comorbidity database entries in a comorbidity database wherein each comorbidity database entry in the plurality of comorbidity database entries includes a comorbidity cross-reference that cross references the one or more tags of a first subset of user generated content files associated with the first group of user profiles with a subset of user profiles from the second group of user profiles that have tangentially related mental health conditions to the one or more tags of the first subset of user generated content files; and
a third server that is configured to publish the first subset of user generated content files to the subset of user profiles.

2. The system of claim 1, wherein the plurality of user generated content files are video files.

3. The system of claim 2, wherein the video files are short form video files between five seconds and ten minutes in length.

4. The system of claim 2, wherein the video files are short form video files between five seconds and two minutes in length.

5. The system of claim 3, wherein engagement of the first subset of user generated content files with the subset of user profiles is a factor of the comorbidity cross-reference.

6. The system of claim 3, wherein a frequency that a first tag of a first mental health condition is used in a single user generated content file with a second tag of a second mental health condition is a factor of the comorbidity cross-reference.

7. The system of claim 3, wherein a level of engagement of the first subset of user generated content files within the first group of user profiles is used to prioritize the first subset of user generated content files to the subset of user profiles.

8. The system of claim 3, wherein demographics are used to prioritize the first subset of user generated content files to the subset of user profiles.

9. The system of claim 3, wherein recency is used to prioritize the first subset of user generated content files to the subset of user profiles.

10. A system for displaying short form video files comprising:
a plurality of user profiles stored in a user database wherein each user profile in the plurality of user profiles is associated with one or more user mental health conditions;
a first server that for each user mental health condition creates at least a first group of user profiles that have user profiles associated with the user mental health condition and a second group of user profiles that are user profiles that are not associated with the mental health condition;
a network of servers with a plurality of user generated short form video files stored thereon, wherein each user generated short form video file in the plurality of user generated short form video files is associated with one or more tags that classifies each user generated short form video file with the one or more mental health conditions;
a second server that for each tag in the one or more tags classifies each user generated short form video file in the plurality of user generated short form video files into the first group of user profiles or the second group of user profiles based on whether a tag in the one or more tags matches the user mental health condition;
a plurality of comorbidity database entries in a comorbidity database wherein each comorbidity database entry in the plurality of comorbidity database entries includes a comorbidity cross-reference that cross references the one or more tags of a first subset of user generated short form video files associated with the first group of user profiles with a subset of user profiles from the second group of user profiles that have tangentially related mental health conditions to the one or more tags of the first subset of user generated short form video files; and
a third server that is configured to publish the first subset of user generated short form video files to the subset of user profiles.

11. The system of claim 10, wherein the user generated short form video files are between five seconds and ten minutes in length.

12. The system of claim 10, wherein the user generated short form video files are between five seconds and two minutes in length.

13. The system of claim 10, wherein engagement of the first subset of user generated short form video files with the subset of user profiles is a factor of the comorbidity cross-reference.

14. The system of claim 10, wherein a frequency that a first tag of a first mental health condition is used in a single user generated short form video file with a second tag of a second mental health condition is a factor of the comorbidity cross-reference.

15. The system of claim 10, wherein a level of engagement of the first subset of user generated short form video files within the first group of user profiles is used to prioritize the first subset of user generated short form video files to the subset of user profiles.

16. The system of claim 10, wherein demographics are used to prioritize the first subset of user generated short form files to the subset of user profiles.

17. The system of claim 10, wherein recency is used to prioritize the first subset of user generated short form video files to the subset of user profiles.

18. A system for displaying video files comprising:
a plurality of user profiles stored in a user database wherein each user profile in the plurality of user profiles is associated with one or more user mental health conditions;
a first server that for each user mental health condition creates at least a first group of user profiles that have user profiles associated with the user mental health condition and a second group of user profiles that are user profiles that are not associated with the mental health condition;
a network of servers with a plurality of user generated video files stored thereon, wherein each user generated video file in the plurality of user generated video files is associated with one or more tags that classifies each user generated video file with the one or more mental health conditions;
a second server that for each tag in the one or more tags classifies each user generated video file in the plurality of user generated video files into the first group of user profiles or the second group of user profiles based on whether a tag in the one or more tags matches the user mental health condition;
a plurality of comorbidity database entries in a comorbidity database wherein each comorbidity database entry in the plurality of comorbidity database entries includes a comorbidity cross-reference that cross references the one or more tags of a first subset of user generated video files associated with the first group of user profiles with a subset of user profiles from the second group of user profiles that have tangentially related mental health conditions to the one or more tags of the first subset of user generated video files; and
a third server that is configured to publish the first subset of user generated video files to the subset of user profiles;
wherein engagement of the first subset of user generated video files with the subset of user profiles is a factor of the comorbidity cross-reference.

19. The system of claim 18, wherein a frequency that a first tag of a first mental health condition is used in a single user generated video file with a second tag of a second mental health condition is a factor of the comorbidity cross-reference.

20. The system of claim 10, wherein a level of engagement of the first subset of user generated video files within the first group of user profiles is used to prioritize the first subset of user generated video files to the subset of user profiles
